(19) United States
(12) Patent Application Publication
(10) Pub. No.: US 2025/0262069 A1
(43) Pub. Date: Aug. 21, 2025

(54) COVERING ELEMENT AND SYSTEM MADE UP OF A COVERING ELEMENT AND AN ORTHOPAEDIC DEVICE

(71) Applicant: OTTO BOCK HEALTHCARE PRODUCTS GMBH, Wien (AT)

(72) Inventors: Daniel SCHATZMAYR, Wien (AT); Peter HILLER, Wien (AT)

(21) Appl. No.: 18/570,311

(22) PCT Filed: Jun. 14, 2022
(86) PCT No.: PCT/EP2022/066208

§ 371 Date: Dec. 14, 2023

(30) Foreign Application Priority Data
Jun. 21, 2021 (DE) 10 2021 115 951.2

(51) Int. Cl. A61F 2/78; A61F 2/50; A61F 2/70

(52) U.S. Cl. CPC A61F 2/78; A61F 2/70; A61F 2002/5001; A61F 2002/5083; A61F 2002/702; A61F 2002/704

(57) ABSTRACT

The invention relates to a covering element for arranging on an orthopaedic device (1), wherein the covering element (10) has a supporting structure (11) which, when the covering element (10) is in the state attached to the orthopaedic device (1), has an inner side (12) facing the orthopaedic device (1) and an outer side (13) facing away from the latter, and at least one fastening device (15) for fastening the covering element (10) to the orthopaedic device (1), wherein at least one photovoltaic cell (20) is arranged on the outer side (13) or so as to be oriented towards the outer side (13).

The invention relates to a covering element to be arranged on an orthopedic device, the covering element comprising a supporting structure which, when the covering element is in the state fitted on the orthopedic device, has an inner side facing toward the orthopedic device and an outer side facing away from the orthopedic device, as well as a fastening device for fixing the covering element on the orthopedic device. The invention likewise relates to a system consisting of such a covering element and an orthopedic device, which is configured to be arranged on a body of a wearer, the orthopedic device comprising at least one electrical or electronic component.

Orthopedic devices such as ortheses, prostheses or exoskeletons are arranged on the body of the respective user. Prostheses replace limbs which are not present or no longer present, or parts thereof, in respect of their external appearance and their functions, insofar as this is possible. Ortheses are arranged on limbs and assist, limit or carry out a movement or hold the limbs in a desired state. In the case of prostheses, the fastening is generally carried out by using a prosthesis socket, which is fastened on a stump. A further option for fastening a prosthesis on a limb consists in an osseointegrated attachment, in which the fastening elements are anchored in a bone. Ortheses are fastened on the respective limb or limb parts by using belts, shells or other bearing surfaces and fastening elements. Exoskeletons are particular embodiments of ortheses.

The functional components of orthopedic devices, for example articulation devices, dampers, actuators, sensors, coupling elements and the like, which need to be protected against environmental effects and which in very rare cases correspond in respect of their optical appearance to the natural limb, may be protected by using coverings.

DE 20 309 318 U1 relates to a knee region partition adapter for detachably connecting a first cosmetic foam part which can be pushed over a knee joint of a prosthetic leg and pulled over a femoral socket. A second cosmetic foam part is arranged around a lower leg part of the leg prosthesis. Two plate-like adapter parts detachably connectable to one another are provided, a first adapter part with the first cosmetic foam part and a second adapter part with the second cosmetic foam part being detachably connectable. The foam part fully encloses the prosthetic joint.

US 2007/0150069 A1 relates to a modularized leg prosthesis cosmetic element having a femoral module which can be fixed on a femoral socket, having a lower leg module which can be fixed on a lower leg tube, and having a patellar module which is pivotably connected to the lower leg module. The cosmetic element, which is obtained by fastening the modules to one another, is covered by a skin module made of a stretchable fiber.

US 2007/0162154 A1 relates to an orthopedic cover for a prosthesis part having a hollow, substantially cylindrical base body for receiving the prosthetic component, having an opening which is large enough for a prosthetic knee joint to be flexed. The width is dimensioned sufficiently to provide protection for the mechanical components of the prosthetic knee joint.

DE 10 2012 009 757 A1 relates to a prosthetic device and a covering for a prosthetic device, in which the outer contour of the prosthetic device is individually adaptable. A rotary module is used to rotate a lower part relative to an upper part of a first part of a prosthetic device about its longitudinal axis. An esthetic covering is thereby intended to be achieved in the rotated state.

U.S. Pat. No. 8,366,789 B1 relates to a prosthetic device having an outer surface, which is configured mirror-symmetrically with respect to the surface of the contralateral healthy leg. The healthy limb is scanned, and the acquired data are processed in order to obtain a corresponding contour for the prosthetic device. The design data of the prosthetic device are processed in the scope of rapid prototyping in order to produce a complete prosthesis, including the outer contour, which corresponds to a healthy leg.

The company Otto Bock markets under the name 4X880 Genium Protective Cover a prosthesis covering having a base body with a continuous surface, which forms a cavity in which a prosthetic knee joint and a lower leg tube can be received. A slot, which can be closed by using insert elements, is formed in the calf region.

It is an object of the present invention to provide a covering element for an orthopedic device and a system consisting of a covering element and an orthopedic device, with which an improved functionality of the orthopedic device can be provided.

This object is achieved by a covering element having the features of the main claim and by a system having the features of the alternative independent claim. Configurations and developments of the invention are disclosed in the dependent claims, the description and the figures.

The covering element to be arranged on an orthopedic device, the covering element comprising a supporting structure which, when the covering element is in the state fitted on the orthopedic device, has an inner side facing toward the orthopedic device and an outer side facing away from the orthopedic device, as well as a fastening device for fixing the covering element on the orthopedic device, is one wherein at least one photovoltaic cell is arranged on the outer side or aligned toward the outer side. By using a solar-assisted current supply, it is possible to provide an improved functionality both of the covering element and of the orthopedic device since electrical consumers can be supplied with current by using the photovoltaic cell. In this way, the utilization time of the orthopedic device is extended or possibilities for the use of a covering element which have not previously been possible are offered. The photovoltaic cell is arranged on the covering element or integrated in the covering element, and is arranged on the covering element so that light can shine on the cell when the covering element is not covered by an article of clothing, or the like. The electrical energy generated by the photovoltaic cell may be stored in an energy storage unit or delivered directly to a consumer. For this purpose, the photovoltaic cell is coupled to an energy storage unit and/or at least one consumer. Both the consumer and the energy storage unit may be arranged on the covering element.

The covering element having the photovoltaic cell is configured in particular as a frame and/or protective casing. A frame encloses functional components such as dampers, articulation devices, terminal elements and similar technical components of the orthopedic device, provides a geometrically stable outer contour and may be a carrier of further components, for example sensors, a control device or the like. During regular operation, the principal mechanical loads of the prosthesis or orthesis are generally not absorbed by the frame. In one configuration, the frame may be configured with sufficient stability to perform a protective function and absorb and forward mechanical loads. For this purpose, the frame at least partially encloses orthesis components or prosthesis components, advantageously fully in certain regions. Alternatively, the covering element is configured as a protective casing which can be arranged around the orthesis components or prosthesis components, or around the frame or on the outer side of the components. The protective casing is arranged on the outer side of the prosthesis component, orthesis component or frame, and is fixed there by using the fastening device. A frame combined with a protective casing may likewise constitute a covering element.

In one configuration, the fastening device for fixing the covering element on the orthopedic device is configured as a form-fit element. As an alternative or in addition, the fastening device is configured as a force-fit element. Examples of form-fit elements are pins, screws, clips, plug elements, bolts, undercuts or a hook and loop fastener; examples of a force-fit element are magnetic retention or a clamping element.

The supporting structure may be configured to be resilient or flexible, so that the supporting structure, and therefore also the photovoltaic cell, can be adapted easily to the orthopedic device. In one configuration, the supporting structure is configured to be geometrically stable and provides an outer contour which may also differ from the outer contour of the orthopedic device. The geometrical stability is ensured by a corresponding design and a corresponding material selection of the supporting structure. A geometrically stable supporting structure may also be bendable. In one alternative, the supporting structure is configured to be foldable and comprises a hinge, for example a film hinge or an articulation having a defined pivot axis, so that two parts of the supporting structure are foldably connected to one another. The supporting structure may be configured with a continuous wall or interrupted. In the case of a configuration with a continuous wall, the photovoltaic cell is preferably arranged on the outer side. If the supporting structure is interrupted, the photovoltaic cell may be arranged inside a recess or interruption, or may be arranged behind the latter. The outer surface of the photovoltaic cell may end flush with the outer side of the supporting structure, or may be arranged set back in order to provide mechanical protection, or may protrude beyond it in order to allow the greatest possible incidence of light. If the supporting structure is translucent, the photovoltaic cell may also be arranged behind the supporting structure.

In one configuration, an energy storage unit, a lighting means, a transmitter device, a receiver and/or an electrical contacting device are arranged on the supporting structure and coupled to the photovoltaic cell. By means of the energy storage unit, it is possible to configure the covering element as a retrofit module and also operate it autonomously, even when the photovoltaic cell is not receiving any incident light. By means of the energy storage unit arranged or integrated in the covering element, a lighting means, a transmitter device for transmitting data or a receiver for receiving data may for example be operated without interruption, at least so long as the energy storage unit is sufficiently charged. The photovoltaic cell may be connected or coupled directly to an electrical contacting device or to the electrical contacting device via the energy storage unit. In this way, it is possible to deliver electrical energy from the photovoltaic cell to a consumer or to another energy storage unit which is arranged on or in the orthopedic device. The covering element may therefore be used either as additional storage for storing electrical energy or for direct energy supply. Furthermore, data may be transmitted via the transmitter device or received via the receiver device, so that for example a positioning signal may be emitted. The lighting means may facilitate orientation or be configured as a coloration element, so that the surface of the covering element may be presented and illuminated in different colors or color gradients.

The electrical contacting device may be configured as a contact face, plug connector or inductive transmission device. The contact face is, in particular, arranged on the inner side of the covering element and positioned in such a way that it lies opposite a corresponding contact face on the orthopedic device, so that electrical contacting takes place when a covering element is fitted. Alternatively, a plug element is coupled to the photovoltaic cell or to an energy storage unit coupled thereto, for example via a cable or an electrical line integrated on or inside the covering element. After the covering element is fitted on the orthopedic device, the plug is then connected to the corresponding plug element on the orthopedic device. Electrical contacting without mechanical coupling takes place via an inductive transmission device, that is to say via a corresponding coil arrangement in the covering element and the orthopedic device.

The photovoltaic cell may be arranged over the entire surface of the covering element. The photovoltaic cell can generate commensurately more electrical energy when its surface area is larger.

The system consisting of a covering element, as has been described above, and an orthopedic device, which is configured to be arranged on a body of a wearer, the orthopedic device comprising at least one electrical or electronic component, is one wherein the electrical or electronic component is electrically conductively connected or connectable to the photovoltaic cell. The connection may take place directly or via an interconnected energy storage unit. An energy storage unit associated by design with the orthopedic device is likewise an electronic component.

The orthopedic device is in particular an orthesis, a prosthesis or an exoskeleton. In one configuration, the covering element is fastened on the orthopedic device in such a way that it can be repeatedly removed. For this purpose, the fastening devices or the fastening device are configured to be repeatedly detachable and attachable. The fastening device may likewise be used to establish electrical contacting between the photovoltaic cell and the orthopedic device. For example, a fastener for securing the covering element on the orthopedic device may simultaneously establish electrical contacting.

In one configuration, the electrical or electronic component is configured as an energy storage unit, data processing device or actuator. The actuator is used, for example, to operate an orthesis or prosthesis, as a device to adjust damping elements, or the like. The data processing device is, for example, provided and configured as a control device for controlling actuators or resistive devices, as well as drives, or for processing sensor data or control data transmitted from an external source, and comprises the components necessary for this purpose, such as microprocessors, energy storage units, software, data memories and the like.

Besides configuration of the actuator as an electrical drive or electronic component, the actuator may alternatively also be configured as a nonelectrical or nonelectronic component.

In one configuration, at least one terminal for an external current source or a data transmission device is arranged on the covering element and/or the orthopedic device, so that energy and/or data can be transmitted to the orthopedic device or the covering element via the covering element or into the orthopedic device.

Exemplary embodiments will be explained in more detail below with the aid of the appended figures. Identical reference signs refer to identical components.

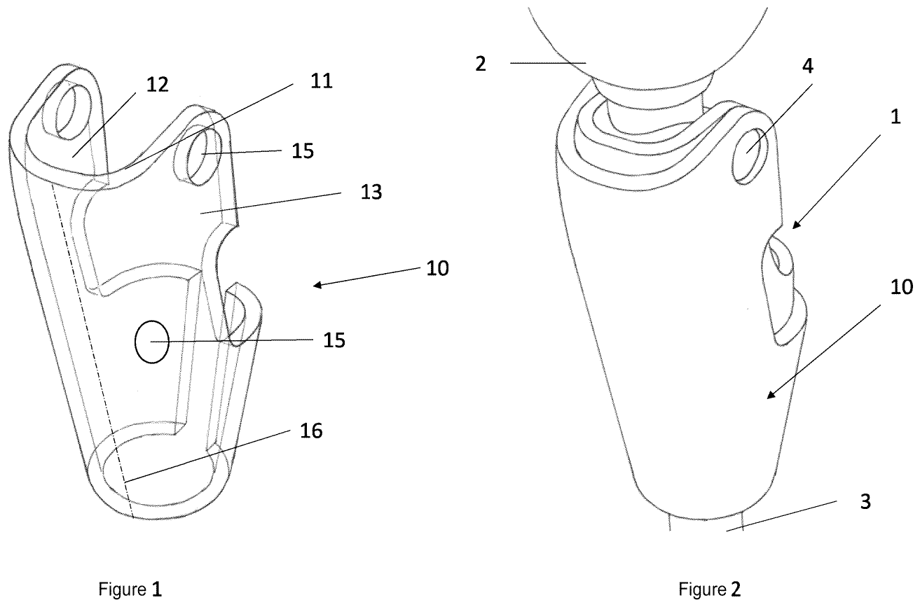

FIG. 1—shows a schematic individual representation of a covering element;

FIG. 2—shows a system consisting of a prosthetic knee joint and a covering element;

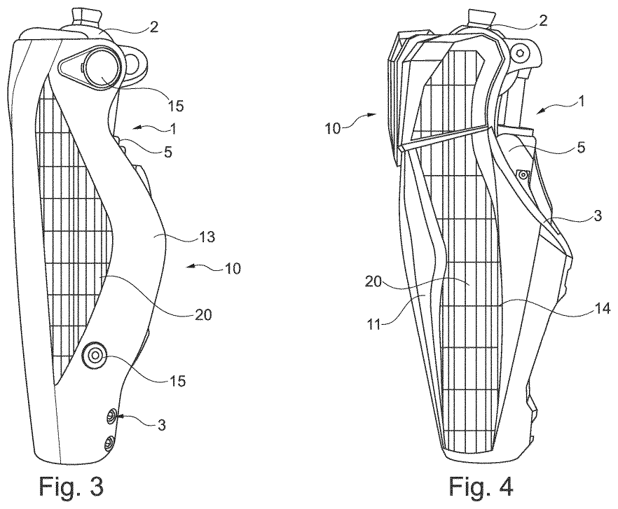

FIG. 3—shows a covering element as a frame;

FIG. 4—shows a covering element as an attachable protective casing;

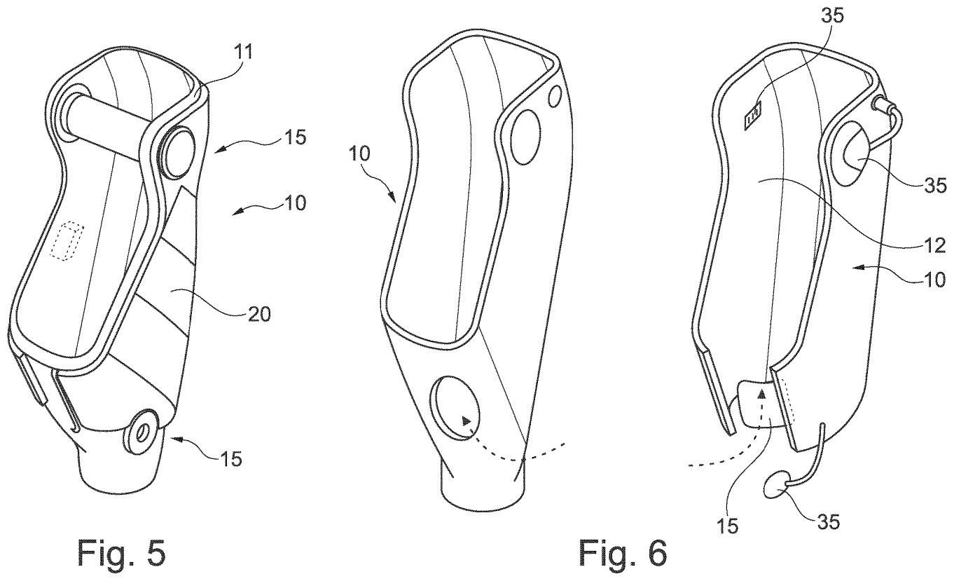

FIG. 5—shows a frame having a photovoltaic cell;

FIG. 6—shows a schematic representation of a combination of a protective casing and a frame;

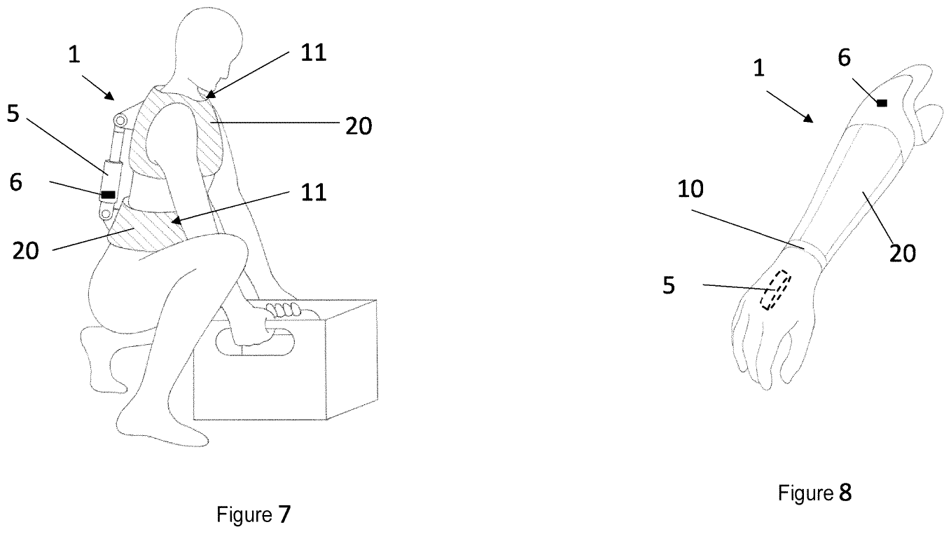

FIG. 7—shows a representation of the orthopedic device as an exoskeleton;

FIG. 8—shows a representation of the orthopedic device as a prosthetic arm;

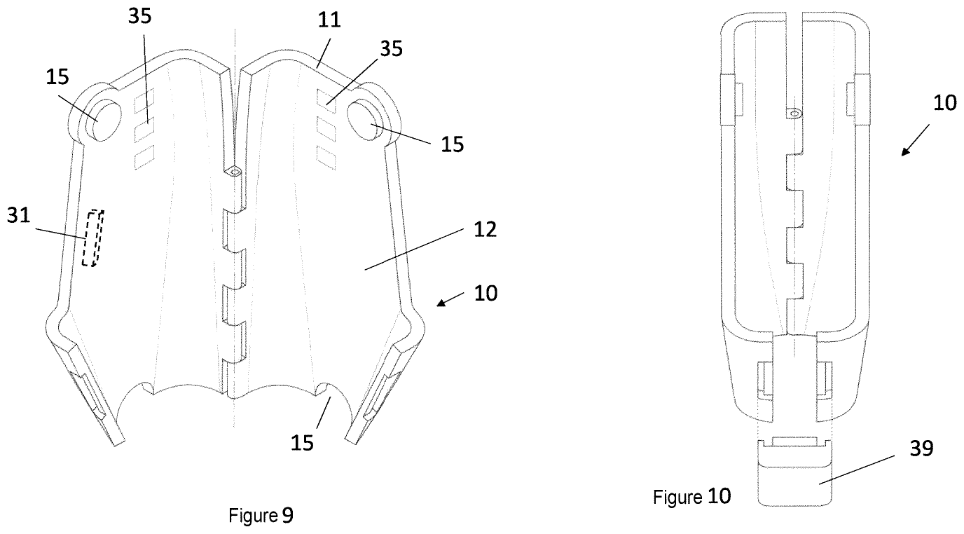

FIG. 9—shows a covering element in the folded apart state;

FIG. 10—shows a covering element according to FIG. 9 with a fastener;

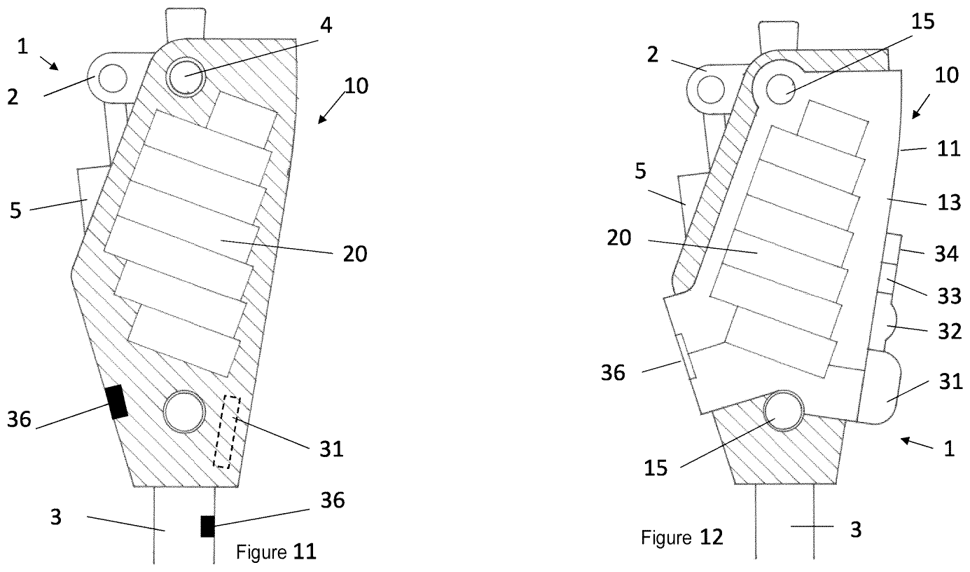

FIG. 11—shows a schematic representation of a frame;

FIG. 12—shows a covering element on a frame;

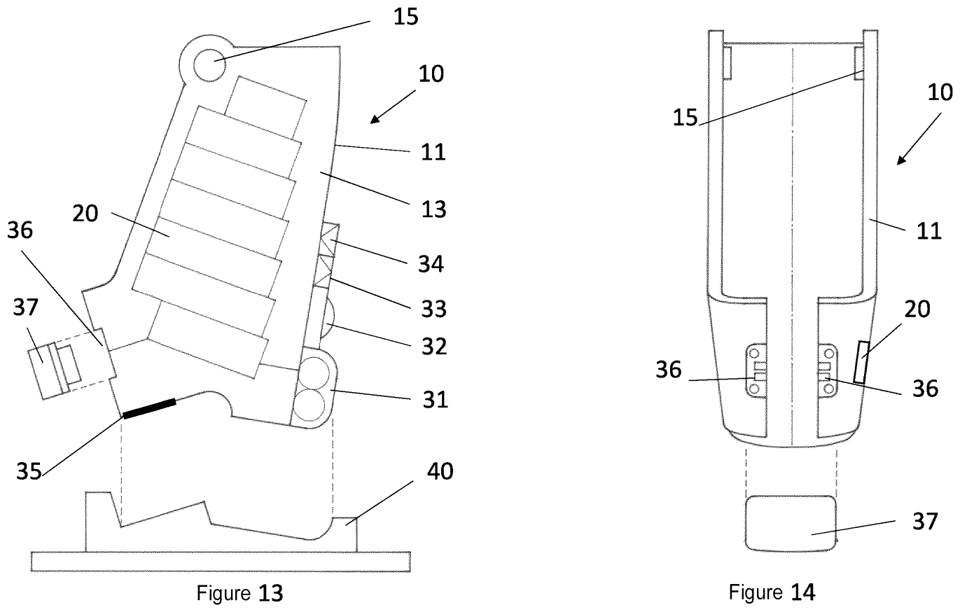

FIG. 13—shows a covering element in a tray;

FIG. 14—shows a covering element with a plug and a jack;

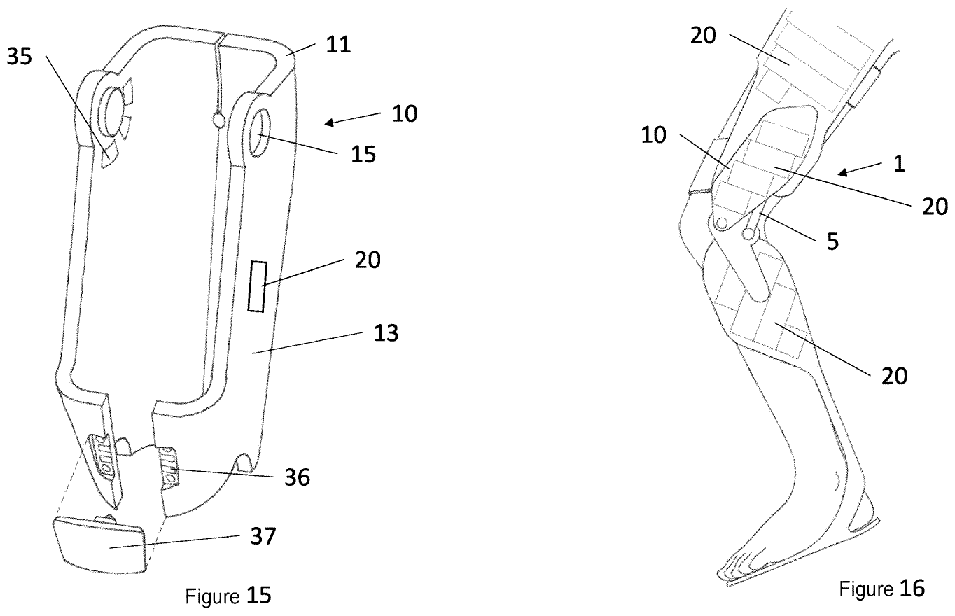

FIG. 15—shows a perspective view of FIG. 14; and

FIG. 16—shows an orthopedic device as an orthesis.

FIG. 1 shows a schematic representation of an individual view of a covering element 10 in the form of a protective casing having a supporting structure 11 which, in the exemplary embodiment represented, is configured as a continuous uninterrupted wall. As an alternative to a continuous wall, the supporting structure 11 may be interrupted. It is likewise conceivable to produce a supporting structure 11 from a plurality of components, for example an outer casing, a central layer and an inner casing. The supporting structure 11 has an inner side 12 and an outer side 13; in a fitted state of the covering element 10, the inner side 12 faces toward the orthopedic device (not represented), while the outer side 13 correspondingly faces away from the orthopedic device. In the exemplary embodiment represented, the covering element 10 is used to cover or enclose a prosthetic knee joint, and forms a cavity in which the prosthetic knee joint may be arranged. The cross section of the covering element 10 is open so that, in order to fit the covering element 10 when a prosthetic knee joint is already in place, the protective casing is bent open, placed around the prosthetic knee joint and then bent back into the initial shape represented. For this purpose, the supporting structure 11 is advantageously configured to be resilient and can be bent open along a bending axis 16. The bending axis 16 extends in the proximodistal direction and lies opposite a slot-like opening on the rear side of the covering element 10. As an alternative to a configuration with a bending axis 16, a design of the covering element 10 in several pieces may also be produced, in which two components of the covering element 10 are pivoted relative to one another about a defined pivot axis in the manner of a hinge.

In order to be able to fix the covering element 10 on the orthopedic device, fastening devices 15 are arranged, or formed, on the supporting structure 11. In the upper region of the covering element 10, two recesses 15 are formed on opposite side walls inside the supporting structure 11 of the covering element 10, into which projections or axial bolts of the prosthetic joint engage and therefore create form-fit locking. In the exemplary embodiment represented, a form-fit element 15 in the form of a magnet, with which it is possible to carry out force-fit fastening of the covering element 10 on the orthopedic device, is positioned in a lower rear region next to the slot-like opening.

As an alternative to the fastening devices 15 represented, they may also be used as straps, buckles, a hook and loop fastener, screws, pins, clips or as combinations thereof. The magnetic pins or spring elements provide both a form-fit and a force-fit fastening option.

A covering element 10 as represented in FIG. 1 may repeatedly be fitted on an orthopedic device, fastened thereon and removed again. The shaping of the covering element 10 is primarily dictated by the shaping of the object to be covered, for example a prosthetic knee joint, a prosthetic ankle joint, a lower leg tube, or an orthesis. On an orthesis, a covering element 10 may for example be arranged on a fastening shell to be fitted on the femur, the lower leg or another body structure. The covering element 10 therefore at least partially covers the orthopedic device to be covered and on the outer side is exposed to incident light, or can be exposed to incident light. A photovoltaic cell (not represented), which will be explained in more detail below, is arranged on the outer side 13 or else on the covering element 10 in a manner accessible from the outer side 13.

FIG. 2 shows the covering element 10 in a state in which it is fitted on an orthopedic device 1. The orthopedic device 1 is configured as a prosthetic knee joint having an upper part 2 and a lower part 3, which are mounted so that they can be pivoted relative to one another about a pivot axis 4. The orthopedic device 1 comprises further components, which will be explained in more detail below, in particular a damping device and optionally electrical and electronic components for controlling the damping behavior of the prosthetic knee joint. As an alternative to a purely passive damping device, a drive may also be arranged in the prosthetic knee joint; sensors, data processing devices, communication interfaces and energy storage units are likewise envisioned as part of the orthopedic device 1. The covering element 10 encloses the orthopedic device 1 almost fully on its outer side, and the inner side 12 of the covering element 10 bears at least pointwise on the outer side of the orthopedic device 1. The outer side 13 of the covering element 10 forms the outer contour of the system consisting of the orthopedic device 1 and the covering element 10. In the exemplary embodiment represented, the upper region of a lower leg is replicated in respect of its shape, and is followed in the distal region by a lower leg tube. A prosthesis socket for receiving a femoral stump is arranged on the upper part 2. If the orthopedic component 1 has a shape that is not desired or additionally needs protection, the covering element 10 may be arranged and fastened on the outer side of the orthopedic device 1.

FIG. 3 shows a variant in which the orthopedic device 1 with the upper part 2 and the lower part 3 is enclosed almost fully by the covering element 10. The orthopedic device 1 comprises a hydraulic damper or actuator 5, which is adjusted situation-dependently by using an electronic controller. The electrical energy required therefor is provided by means of an energy storage unit (not represented), which needs to be charged regularly. Arranged on the outer side 13 of the covering element 10, which in the exemplary embodiment represented is configured as a geometrically stable frame, there is a photovoltaic cell 20 which extends over a large part of the outer side 13. Arranged on the opposite side of the covering element 10, there is also a corresponding photovoltaic cell which, when light is incident, generates electrical energy that is stored in an energy storage unit (not represented). An energy storage unit may be arranged in the covering element 10 and/or in the orthopedic device 1. The photovoltaic cell 20 is connected to the energy storage unit via suitable electrical lines and electrical contacting devices such as plugs, contact faces, inductive transmission devices or the like. The frame is fixed as a covering element 10 by means of mechanical fastening devices 15.

FIG. 4 shows a variant in which an orthopedic device 1 in the form of a prosthetic knee joint is likewise enclosed by a covering element 10. The covering element 10 is configured as a protective casing, as schematically represented in FIG. 1, and is fitted on the prosthetic knee joint as an orthopedic device 1 and is held with a form fit and/or clamping by being bent open and springing back. The fastening may be assisted by magnets or additional fastening devices, which are not represented. The supporting structure 11 on the protective casing as a covering element 10 is provided with a recess 14, inside which the photovoltaic cell 20 is arranged. The photovoltaic cell 20 may be arranged set back behind the outer side of the supporting structure 11 or may end flush with the outer side of the supporting structure 11. The electrical energy generated by the photovoltaic cell 20 is supplied to a consumer and/or an energy storage unit.

FIG. 5 represents a schematic representation of the covering element 10 as a frame, on the outer side of which the photovoltaic cell 20 is arranged. The photovoltaic cell 20 may occupy the entire outer side of the frame 10 and be fixed on the outer side of the frame with a form fit by using fastening devices 15 and/or with a force fit and/or materially by adhesive bonding or other fastening devices. Formed inside the frame and inside the supporting structure 11, there is a cavity for receiving the orthopedic device 1, which provides the functionality of the orthopedic device 1. The frame is used as a geometrically stable casing and also forms the outer contour. Additional components for the orthopedic device 1 may be arranged inside the frame, for example sensors, interfaces for data exchange and/or energy exchange, control devices and/or energy storage units. A GPS module, a transmitter device and/or a receiver device for the wireless exchange of information may likewise be formed or arranged on the frame. In the exemplary embodiment, forwarding of mechanical forces from the upper part 2 to the lower part 3 via the frame does not take place, or does not take place to a significant extent.

FIG. 6 represents a variant in which the frame is configured together with the protective casing as a covering element 10. The protective casing as part of the system with the frame, which together form the covering element 10, may be pressed on, adhesively bonded, screwed, held by using magnets or form-fit elements on the outer side of the frame or held thereon by clamping or using a combination of the aforementioned components and devices. The outer side of the protective casing 10 bears at least one photovoltaic cell 20. The photovoltaic cell 20 forwards the energy via electrical contacting devices 35 to an electrical or electronic component of the orthopedic device 1, optionally with the interconnection of the frame. For this purpose, contact faces as a contacting device 35 are arranged on the inner side 12 and are positioned so as to correspond with contact faces arranged on the outer side either of the frame or of the orthopedic device 1. In the fitted state, there is thus an electrical connection between the photovoltaic cell 20 and the electrical or electronic component of the orthopedic device 1. As an alternative or in addition, a cable is arranged on the lower side of the protective casing 10, arranged on the end of which there is an electrical plug as a contacting device 35 which can be inserted into a corresponding jack either on the frame or via a through-opening directly into the orthopedic device and can be connected to the electrical or electronic component. As an alternative or in addition, the energy transmission may take place via an induction coil 35 which, in the exemplary embodiment represented, is arranged on the upper end of the protective casing 10.

The protective casing 10 encloses the frame almost fully in the fitted state and can be secured on the rear side by using a strap or a fastener 15, so that after the covering element 10 is folded apart or bent open, it is held firmly on the frame or the orthopedic device.

The covering element, which is configured as a protective casing and/or as a frame, may comprise further components besides an energy storage unit, so that an extended functionality is ensured together with energy independence. The further components are for example a lighting device, which may be used as an emergency signal. Emergency call devices in combination with positioning systems may likewise be an integrated part of the covering element 10. In some embodiments, terminal devices for connecting to external instruments, data sources or energy sources are arranged on the outer side or externally accessibly, so that the orthopedic device may be charged or supplied with data, or exchange of data and/or energy may take place with the external instrument without the covering element 10 having to be taken off.

FIG. 7 shows a further embodiment of the orthopedic device 1 in the form of an exoskeleton which is fitted on the torso of a user. The exoskeleton comprises two components which are fastened on a patient, one in the abdominal region and the other in the shoulder region. Fastened on the outer side of the components, which form the supporting structures 11, there are photovoltaic cells 20 by means of which light is converted into electrical energy. The two components are connected to one another by means of an actuator 5, to which an electronic control element 6 is assigned. The actuator 5 may comprise a motor or may operate or adjust a damping device or braking device, and is supplied with the necessary signals by means of the control device 6. The photovoltaic cells 20 are connected both to the actuator 5 and to the electronic control element 6, and on the basis of sensor data perform an adjustment either of the position or of the resistance between the two components of the exoskeleton.

A further embodiment of the orthopedic device 1 is represented in FIG. 8, which shows a prosthetic arm having a prosthesis socket and a prosthetic hand. Arranged as an actuator 5 inside the prosthetic hand, there is a motor which is activated or deactivated by means of a control device (not represented) on the basis of control signals, for example myoelectrical signals from the upper arm. By using the actuator 5, the prosthetic fingers may be adjusted or the hand may be displaced relative to the forearm. A covering element 10 with photovoltaic cells 20 arranged on its outer side is fastened on the outer side of the forearm socket 1. The fastening may be carried out permanently or in particular reversibly, for example by using a magnetic fastener, form-fit elements such as a hook and loop fastener, screws, clip elements or the like. If the covering element 10 has a sufficient structural strength, fitting on the outer side of the prosthesis socket may increase the strength and stability of the orthopedic device 1.

FIG. 9 shows a perspective representation of the covering element 10 as a foldable covering element consisting of two lateral shells in a folded apart state. The two lateral shells can be folded apart and folded together along a pivot axis which, in the exemplary embodiment represented, runs inside the front wall. The pivot axis may also be arranged, or formed, at places other than this. Contact faces or plug elements 35 are arranged on the inner side 12 of the covering element 10 in order to send electrical energy from photovoltaic cells, which are arranged on the outer side of the lateral shells, to the orthopedic device (not represented). An energy storage unit 31 is schematically indicated and accommodated inside the supporting structure 11 of one lateral shell. If the energy delivered by the photovoltaic cells is not immediately needed, temporary storage may take place in the energy storage unit 31. In the folded apart state, the covering element 10 is fitted around the respective orthopedic component, for example a prosthetic knee joint, and fixed thereon by means of the fastening devices 15. Similarly as in FIG. 5, the fastening devices are configured as form-fit elements. Semicircular recesses are formed as a fastening element or form-fit element 15 at the lower, distal end of the covering element 10, while studs, optionally magnetic studs 15, engage in corresponding recesses of the orthopedic device at the proximal, upper end. In the closed state, the contact faces 35 enter into electrical contact with corresponding contact faces on the orthopedic device.

FIG. 10 shows the covering element 10 according to FIG. 9 in a folded together state. Recesses and undercuts, into which a fastener 39 can be inserted, are arranged or formed on the rear side of the covering element 10. By means of the fastener 39, the two shells are secured with a form fit on the rear side of the covering element 10. The securing element 39 may also have or fulfill further functions; for example, a sensor, GPS module, energy storage unit or the like may be integrated inside the securing element 39 and then be used in combination with the photovoltaic cells 20 and the orthopedic device.

FIG. 11 shows a configuration of the orthopedic device 1 in the form of a prosthetic knee joint having an upper part 2 and a lower part 3, which is mounted on the upper part 2 in such a way that it can be pivoted about a pivot axis 4. The upper part 2 is displaced relative to the lower part 3, or is influenced in the pivoting movement, by means of an actuator 5. The covering element 10 is configured as a frame, similarly as in the left representation of FIG. 6. Photovoltaic cells 20 are fitted on the outer side of the covering element 10. An energy storage unit 31 and a terminal 36 for an external current source or an external data transmission device are arranged inside the covering element 10. Such a terminal 36 for an external current source or an external data transmission device may also be arranged in the orthopedic device 1, here on the lower part 3. Electrical contacting with the photovoltaic cells 20, or the energy storage unit 31 which is connected to the photovoltaic cells 20, takes place via contact faces on the inner side of the frame 10.

FIG. 12 represents a further variant, in which the covering element 10 is pressed or fixed in another way on the frame around the orthopedic device 1. The covering element 10 has an electrical terminal 36 on its rear side and is fixed, in particular reversibly fixed, on the frame by means of fastening elements 15, as described in the preceding figures. On the outer side 13 of the supporting structure 11, the photovoltaic cells 20 are fastened and electrically connected to an energy storage unit 31 which is arranged on the front side of the covering element 10. Likewise arranged on the covering element 10 are a lighting means 32, a transmitter device 33 and a receiver device 34, which are electrically coupled to the photovoltaic cells 20 directly or via the energy storage unit 31. Electrical contacting 35 on the inner side of the covering element is not indicated for reasons of clarity. The covering element 10 may be configured as a module and configured to be foldable or bendable, in order subsequently to be fixed on the orthopedic device 1, in particular on the frame such as is represented in FIG. 11. In particular, electrical contacting with the orthopedic device need not take place in a modular configuration.

A covering element 10 such as is represented in FIG. 12 is shown as a separate module in FIG. 13. The covering element 10 is located on, or above, a tray 40 in which the covering element 10 may be stowed. The tray 40 may be provided with a charging device, which enters into contact with contact faces 35 on the lower side or the inner side of the covering element 10 as soon as the covering element 10 has been stowed in the tray 40. FIG. 13 shows an external current source 37 in the form of a separate accumulator, plug or battery, which can be fitted to the terminal 36 on the rear side of the covering element 10. Instead of a current source or an energy storage unit, data transmission devices or other external instruments 37 may be arranged on the correspondingly configured terminal device or the terminal 36 and coupled to the electrical and/or electronic components of the covering element 10, and therefore also to the orthopedic device.

FIG. 14 shows a rear view of a covering element 10 according to FIG. 13. The terminals 36 for electrical contacting with the energy storage unit or the external instrument 37 are indicated on the rear side walls. By means of this external instrument or the energy storage unit 37, besides electrical connection via the terminals 36, mechanical securing may also be achieved by a form fit or force fit, so that a combination of mechanical securing, electrical contacting and supplementing with additional functions is possible.

FIG. 15 shows a perspective representation of such a configuration, in which the covering element 10 is bendable. On the rear side, the terminals 36 are arranged on both sides of a slot in the back walls. Likewise visible above and below the electrical contacts or terminals 36 are bores or magnets, by means of which force-fit or form-fit securing to the external instrument 37 is possible with magnets, ferromagnetic components or studs.

FIG. 16 shows a further configuration of the orthopedic device 1 in the form of an orthesis. A foot plate with a lower leg part is mounted in an articulated fashion on a femoral part. By means of suitable fastening elements, for example belts or shells, the orthesis is fixed on the leg of the user. Photovoltaic cells 20 are fastened on the outer side of the lower part and of the upper part. A covering element 10 on the femoral part is detachably connected thereto. Arranged between the femoral part and the lower leg part is the actuator 5, which as an electrical component is supplied with electrical energy by means of the photovoltaic cells 20, or the energy supply is assisted by using the latter.

1. A covering element to be arranged on an orthopedic device, comprising:
a supporting structure which, when the covering element is in a state fitted on the orthopedic device, has an inner side facing toward the orthopedic device and an outer side facing away from the orthopedic device;
at least one fastening device for fixing the covering element on the orthopedic device; and
at least one photovoltaic cell which is either arranged on the outer side of the supporting structure or aligned toward the outer side of the supporting structure.

2. The covering element as claimed in claim 1, wherein the covering element is configured as a frame and/or protective casing.

3. The covering element as claimed in claim 1 wherein the at least one fastening device is configured as a form-fit element and/or force-fit element.

4. The covering element as claimed in claim 1 wherein the supporting structure is configured to be resilient or flexible.

5. The covering element as claimed in claim 1 wherein the supporting structure is configured to be geometrically stable and/or foldable.

6. The covering element as claimed in claim 1 wherein the at least one the photovoltaic cell is arranged on either the outer side of the supporting structure, in the supporting structure, or behind a recess in the supporting structure.

7. The covering element as claimed in claim 1 further comprising at least one an energy storage unit, a lighting means, a transmitter device, a receiver device, and an electrical contacting device is arranged on the supporting structure and coupled to the at least one photovoltaic cell.

8. The covering element as claimed in claim 7, wherein the electrical contacting device is configured as a contact face, plug connector, or inductive transmission device.

9. A system, comprising:
a covering element as claimed in claim 1; and
an orthopedic device, which is to be arranged on a body of a wearer, the orthopedic device comprising at least one electrical or electronic component electrically conductively connected or connectable to the at least one photovoltaic cell.

10. The system as claimed in claim 9, wherein the orthopedic device is a prosthesis, an orthesis, or an exoskeleton.

11. The system as claimed in claim 9, the covering element is fastened on the orthopedic device in such a way that it is repeatedly removable.

12. The system as claimed in claim 9 wherein the at least one electrical or electronic component is configured as an energy storage unit, data processing device’ or actuator.

13. The system as claimed in claim 9, further comprising at least one terminal for an external current source or a data transmission device, wherein the at least one terminal is arranged on the covering element and/or the orthopedic device.

14. The system as claimed in 9 wherein the covering element is configured as a module.
